(19) United States
(12) Patent Application Publication
(10) Pub. No.: US 2015/0320534 A1
(43) Pub. Date: Nov. 12, 2015

(54) FIXING DEVICE FOR INHALATION ANESTHESIA OF RODENTS

(71) Applicant: SAMSUNG ELECTRONICS CO., LTD., Suwon-si, Gyeonggi-do (KR)

(72) Inventor: Geunho IM, Suwon-si (KR)

(21) Appl. No.: 14/652,226

(22) PCT Filed: Nov. 6, 2013
(86) PCT No.: PCT/KR2013/009993

§ 371 Date: Jun. 15, 2015

(30) Foreign Application Priority Data
Dec. 13, 2012 (KR) 10-2012-0145717

(51) Int. Cl. A61D 7/04; A61B 5/097; A61M 16/08; A61M 16/01

(52) U.S. Cl. CPC A61D 7/04; A61M 16/01; A61B 5/097; A61M 16/0841; A61B 2503/40; A61B 2503/42

(57) ABSTRACT

A device for fixing a laboratory rodent for inhalation anesthesia includes: a body including a cavity into which at least a part of a rodent including the nose of the rodent is insertable; and a gas supply pipe inserted into the cavity for supplying an anesthetic gas to the rodent, the gas supply pipe including a fixing part on an end portion thereof for fixing incisors of the rodent and an opening formed at a position forward from the fixing part for discharging the anesthetic gas. Therefore, the position of the opening relatively to the nose of the rodent may be constantly maintained.

### TECHNICAL FIELD

The present disclosure relates to a fixing device for inhalation anesthesia of rodents, the fixing device being configured to stably maintain anesthesia by preventing separation of an anesthetic supply device during inhalation anesthesia.

### BACKGROUND ART

Laboratory rodents such as rats and mice are widely used for preclinical animal testing. Laboratory rodents are anesthetized for experiments such as animal model operations, medicine administration experiments, tissue extraction experiments, or image capturing experiments. Inhalation anesthesia is widely used as a method for anesthetizing rodents.

In the related art, inhalation anesthesia is carried out by putting a tool shaped like a nose cone or a face mask on the head of a laboratory rodent, and an anesthetic is injected through the tool. However, since such a tool is simply put on the head of a laboratory rodent, the tool may be detached when the laboratory rodent moves, and thus anesthesia may not be stably maintained.

In addition, since inhalation anesthesia devices of the related art are only used to anesthetize laboratory rodents, the current state of breathing of laboratory rodents cannot be observed, and thus there is a limit to obtaining correct experimental results.

Furthermore, inhalation anesthesia devices of the related art are required to have supplementary functions for protecting experimenters from odors of anesthetic gases and chemical pollution and reducing laboratory pollution.

### DETAILED DESCRIPTION OF THE INVENTION

### Technical Problem

A fixing device for inhalation anesthesia of rodents is provided. When a rodent is anesthetized by an inhalation method, the fixing device stably maintains the posture of the rodent for continuous anesthetizing of the rodent, and separation of the fixing device from the nose of the rodent is prevented.

In addition, the fixing device for inhalation anesthesia of rodents makes it possible to monitor the state of a rodent during inhalation anesthesia.

In addition, the fixing device for inhalation anesthesia of rodents is configured to minimize leakage of an anesthetic gas.

### Technical Solution

According to an aspect, there is provided a device for fixing a laboratory rodent for inhalation anesthesia, the device including: a body including a cavity into which at least a part of a rodent including the nose of the rodent is insertable; and a gas supply pipe inserted into the cavity for supplying an anesthetic gas to the rodent, the gas supply pipe including a fixing part on an end portion thereof for fixing incisors of the rodent and an opening formed at a position forward from the fixing part for discharging the anesthetic gas, wherein the position of the opening relatively to the nose of the rodent is constantly maintained.

A lower portion of the cavity in which the fixing part and the opening are located may have a diameter greater than that of an opposite upper portion of the cavity.

The lower portion of the cavity may have a cone shape.

A packing member may be provided on a lower portion of the cavity in which the fixing part and the opening are located, so as to make tight contact with the rodent and prevent leakage of the anesthetic gas, exhaled gas of the rodent, and remaining gas.

A flat support surface may be formed on at least a portion of the body.

The device may further include a fixing member to detachably fix the gas supply pipe to the body.

The opening and the fixing part may be provided as parts of a replaceable tip that is attachable to the gas supply pipe and detachable from the gas supply pipe.

The device may further include a discharge tube connected to the cavity for drawing in exhaled gas of the rodent or remaining gas and discharging the exhaled gas and the remaining gas to an outside area.

A discharge pipe of the discharge tube may extend to a lower portion of the cavity.

A connector may be provided on an end portion of the discharge tube for connection with a monitoring device that monitors breathing of the rodent.

### Advantageous Effects

As described above, the fixing device maintains the posture of a rodent when the rodent is anesthetized by an inhalation method, and thus the state of anesthesia may be stably maintained.

In addition, since exhaled gas of a rodent and remaining gas are discharged through the additional discharge pipe, the variations of the amount of carbon dioxide contained in exhaled gas may be measured for monitoring the state of the rodent during anesthesia, and thus more accurate test results may be obtained.

In addition, leakage of an anesthetic gas may be minimized for maintaining a laboratory at a comfortable state and allowing experimenters to stably perform experiments.

### DESCRIPTION OF THE DRAWINGS

FIG. 1 is a perspective view illustrating a fixing device for inhalation anesthesia of rodents according to an exemplary embodiment.

FIG. 2 is an exploded view illustrating the fixing device for inhalation anesthesia of rodents illustrated in FIG. 1, according to an exemplary embodiment.

FIG. 3 is a plan view illustrating the fixing device for inhalation anesthesia of rodents illustrated in FIG. 1, according to an exemplary embodiment.

FIG. 4 is a bottom view illustrating the fixing device for inhalation anesthesia of rodents, the fixing device further including a packing part according to an exemplary embodiment.

### MODE FOR INVENTION

Hereinafter, a fixing device for inhalation anesthesia of rodents will be described in detail with reference to the accompanying drawings according to exemplary embodiments of the present invention. In the drawings, like reference numerals denote like elements. Moreover, detailed descriptions related to well-known functions or configurations will be ruled out in order not to unnecessarily obscure subject matters of the exemplary embodiments.

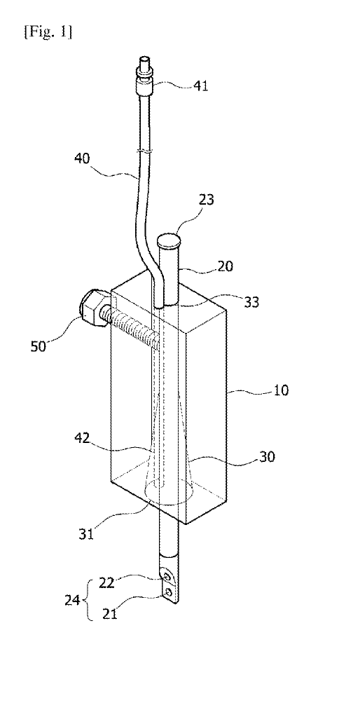

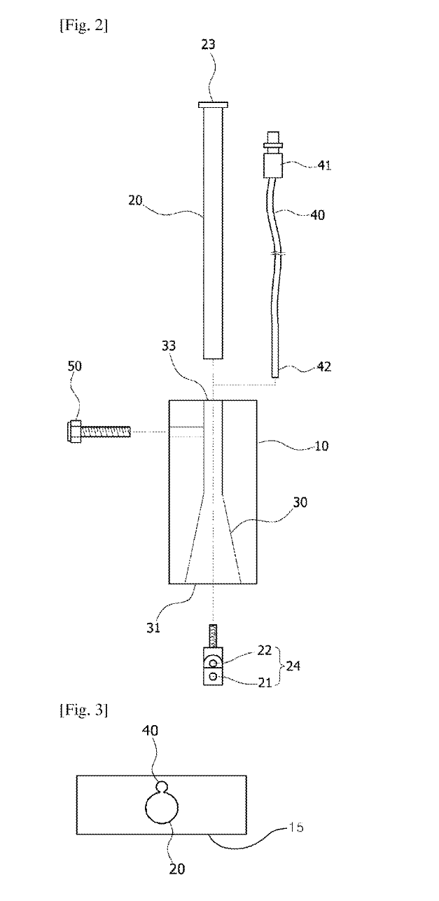

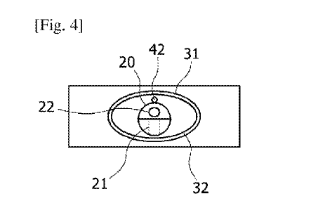

FIG. 1 is a perspective view illustrating a fixing device 100 for inhalation anesthesia of rodents according to an exemplary embodiment. FIG. 2 is an exploded view illustrating the fixing device 100 for inhalation anesthesia of rodents illustrated in FIG. 1, according to an exemplary embodiment, and FIG. 3 is a plan view illustrating the fixing device 100 for inhalation anesthesia of rodents illustrated in FIG. 1, according to an exemplary embodiment. FIG. 4 is a bottom view illustrating the fixing device 100 for inhalation anesthesia of rodents, the fixing device 100 further including a packing part according to an exemplary embodiment.

The fixing device 100 for inhalation anesthesia may include a body 10 in which a cavity 30 is formed, and a gas supply pipe 20 configured to supply an anesthetic gas from a supply unit to the nose of a rodent.

For example, the body 10 may be formed of a transparent acrylic material. However, the present invention is not limited thereto. For example, the body 10 may be manufactured by any method such as machining or injection molding. Although the shape of the body 10 is not limited to a particular shape, a flat support surface 15 (refer to FIG. 3) may be formed on at least an outer portion of the body 10 so that the body 10 may easily be supported on a testing bench for anesthetizing a rodent. For example, the body 10 may have a rectangular shape, a circular shape, or an elliptical shape partially having a flat support surface 15. Since the body 10 has the flat support surface 15, even though the fixing device 100 is moved by the movement of a rodent during anesthesia, the posture of the rodent may be properly maintained.

The cavity 30 is formed in the body 10. The gas supply pipe 20 is inserted into the cavity 30. An upper portion 33 of the cavity 30 may have a diameter slightly greater than the diameter of the gas supply pipe 20 so that the gas supply pipe 20 may be moved to some degree in the length direction thereof. A lower portion 31 of the cavity 30 is a portion to be put on the head of a rodent including the nose of the rodent, and thus the shape and size of the lower portion 31 may be determined according to the nose and head shapes of rodents to be anesthetized. The lower portion 21 of the cavity 30 may have a diameter greater than that of the upper portion 33 of the cavity 30. In the exemplary embodiments, the cavity 30 has a circular sectional shape. However, the cavity 30 may have a non-circular sectional shape, and in this case, the term “diameter” may be construed as an equivalent diameter. In the exemplary embodiments, the cavity 30 includes the upper portion 33 having a cylinder shape with a circular cross section and the lower portion 31 having a cone shape with an elliptical cross section. That is, the cavity 30 is generally shaped like a funnel. However, the shape and size of the cavity 30 is not limited as long as the gas supply pipe 20 is insertable into the cavity 30 and a part of a rodent such as the head or body of the rodent including the nose is insertable into the cavity 30.

The gas supply pipe 20 is inserted into the cavity 30 of the body 10 for supplying an anesthetic gas from an anesthetic gas supply unit (not shown) to the nose of a rodent. The gas supply pipe 20 may have a hollow pipe shape so that an anesthetic gas may be supplied through the inside of the gas supply pipe 20.

A connection part 23 is provided on an end portion of the gas supply pipe 20 for connection with the anesthetic gas supply unit. The connection part 23 may have a connection structure such as a threaded connection structure for connection with the anesthetic gas supply unit. In this case, for example, the connection part 23 may have a female or male screw, and the anesthetic gas supply unit may have a corresponding male or female screw. Although not illustrated in the drawings, the connection part 23 may be directly connected to the anesthetic gas supply unit or directly connected to the anesthetic gas supply unit through a part such as a tube. The connection structure is not limited to a threaded connection structure. For example, the anesthetic gas supply unit and the connection part 23 may be connected through other connection structures such as a connection structure using an elastic packing member (not shown) provided on the anesthetic gas supply unit or the connection part 23, or a connection structure using a clamp.

The other end portion of the gas supply pipe 20 has an opening 22 so that an anesthetic gas supplied through the gas supply pipe 20 may reach the nose of a rodent through the opening 22. In addition, a fixing part 21 is formed on the other end portion of the gas supply pipe 20 to hold the incisors of a rodent. The fixing part 21 may have a hole shape into which the incisors of a rodent are insertable. Generally, the nose of a rodent protrudes forward from the incisors of the rodent, and thus the distance between the opening 22 and the fixing part 21 may be determined based on the distance between the nose and incisors of rodents.

If the gas supply pipe 20 is repeatedly used, the opening 22 may be clogged with foreign objects, or the fixing part 21 may be broken by the incisors of rodents. In addition, according to the size of a rodent to be anesthetized, another gas supply pipe 20 having a suitable distance between an opening 22 and a fixing part 21 may be required. In this case, the gas supply pipe 20 may be replaced. To this end, the gas supply pipe 20 may be detachably coupled to the body 10.

Referring to FIGS. 1 and 2, the fixing device 100 for inhalation anesthesia may further include a fixing member 50 so as to fix the gas supply pipe 20 to the body 21. After the gas supply pipe 20 is inserted into the cavity 30, the fixing member 50 may be coupled to the body 10 for fixing the gas supply pipe 20 to the body 10. In the exemplary embodiments, the fixing member 50 is coupled to the body 10 by screwing the fixing member 50 through a penetration hole formed from a lateral side to the inside of the body 10 so that an end portion of the fixing member 50 may push the gas supply pipe 20 against a wall of the cavity 30, for example, a wall of the upper portion 33 so as to fix the gas supply pipe 20 to the body 10. The fixing member 50 is not limited to the example illustrated in FIGS. 1 and 2. For example, the fixing member 50 may be a wedge insertable between the cavity 30 and the gas supply pipe 20 or a clamp supported on the body 10.

An explanation will now be given of how a rodent is anesthetized using the fixing device 100.

The fixing member 50 is pulled backward from the cavity 30 of the body 10 so as to smoothly insert the gas supply pipe 20 into the cavity 30, and then the gas supply pipe 20 is inserted into the cavity 30. The incisors of a rodent are inserted into the fixing part 21 of the gas supply pipe 20 which is located close to the lower portion 31 of the cavity 30. The gas supply pipe 20 is pushed in a direction toward the upper portion 33 so as to bring the head or trunk of the rodent into tight contact with the lower portion 31 of the cavity 30 or an inner wall of the lower portion 31. Thereafter, the gas supply pipe 20 is fixed to the body 10 using the fixing member 50 so that the gas supply pipe 20 may not be moved inside the cavity 30.

The connection part 23 is connected to the anesthetic gas supply unit (not shown), and an anesthetic gas is supplied through the gas supply pipe 20. After the anesthetic gas flows through the gas supply pipe 20, the anesthetic gas is discharged through the opening 22. In the state in which the incisors of the rodent are inserted into the fixing part 21, the opening 22 is located in front of the nose of the rodent. Although the rodent moves to some degree before or during anesthesia, the distance between the opening 22 and the nose of the rodent may be constantly maintained. Therefore, the anesthetic gas may be effectively supplied to the nose of the rodent, and thus the state of anesthesia may be stably maintained. In addition, since the gas supply pipe 20 is fixed to the body 10 in a state in which the head or trunk of the rodent is brought into tight contact with the lower portion 31 of the cavity 30 or the inner wall of the lower portion 31, the possibility of leakage of the anesthetic gas may be decreased.

As described above, if the gas supply pipe 20 is repeatedly used, the opening 22 may be clogged with foreign objects, and the fixing part 21 may be broken by the front teeth of a rodent. In addition, another gas supply pipe 20 having a suitable distance between an opening 22 and a fixing part 21 may be necessary according to the size of a rodent. In those cases, only the opening 22 and the fixing part 21 may be replaced instead of entirely replacing the gas supply pipe 20. To this end, as shown in FIGS. 1 and 2, the opening 22 and the fixing part 21 may be provided as parts of a replaceable tip 24. The replaceable tip 24 may be detachably coupled to the other end portion of the gas supply pipe 20. For example, the replaceable tip 24 may be coupled to the other end portion of the gas supply pipe 20 using a threaded structure.

According to the above-described configuration, if the opening 22 is clogged, the fixing part 21 is broken, or it is necessary to change the distance between the opening 22 and the fixing part 21, only the replaceable tip 24 may be replaced. Therefore, the fixing device 100 for inhalation anesthesia may be more effectively used. Referring to FIG. 4, a packing member 32 may be disposed on the lower portion 31 of the cavity 30. For example, the packing member 32 may be a rubber ring or a felt ring. The packing member 32 may be brought into tight contact with the head or trunk of a rodent. In this case, the possibility of leakage of an anesthetic gas from the cavity 30 may be further reduced. Thus, laboratories or experimenters may be effectively protected from contamination, and anesthesia may be maintained more stably.

It is necessary to maintain rodents at a stable state during anesthesia so as to obtain reliable results from experiments performed using the rodents. To this end, according to an exemplary embodiment, the fixing device 100 for inhalation anesthesia may be configured such that the state of breathing of a rodent may be monitored using the fixing device 100. Referring to FIGS. 1 to 4, the fixing device 100 may further include a gas discharge tube 40. The gas discharge tube 40 includes a discharge pipe 42. The discharge pipe 42 may be inserted into the cavity 30 through a penetration hole formed in the upper portion 33 of the body 10. For example, the discharge pipe 42 may have an inner diameter within the range of about 1.5 mm to about 2.0 mm. For stably discharging exhaled gas of a rodent or remaining gas, the discharge pipe 42 may be inserted in such a manner that an inner end portion of the discharge pipe 42, that is, an end portion of the discharge pipe 42 inserted into the cavity 30, is located in the vicinity of the nose of a rodent. In this case, the rate of breathing of a rodent or ETCO₂ may be more reliably measured. A connector 41 may be provided on the other end of the discharge pipe 42, that is, an end portion of the discharge pipe 42 located outside the body 10, so as to connect the discharge pipe 42 to a breathing monitoring device (not shown).

According to this configuration, during anesthesia or testing, exhaled gas of a rodent or remaining gas may be effectively discharged, and thus the state of anesthesia may be stably maintained. In addition, exhaled gas of a rodent may be sent to the monitoring device for measuring the rate of breathing or ETCO₂, and thus correct or more accurate test results may be obtained. Although not shown in the drawings, the sizes and shapes of elements such as the body 10, the cavity 30, the gas supply pipe 20, and the fixing part 21 may be varied according to the kinds of rodents to be anesthetized, such as rats, neonatal rats, or mice.

While the present invention has been shown and described above, it will be apparent to those skilled in the art that modifications and variations could be made without departing from the scope of the present invention as defined by the appended claims. The exemplary embodiments of the present disclosure are for illustrative purposes only and are not intended to limit the scope of the present invention. Therefore, the scope of the invention is defined not by the detailed description but by the appended claims, and all differences within the scope will be construed as being included in the present invention.

### INDUSTRIAL APPLICABILITY

The fixing device may be used for inhalation anesthesia of rodents.

1. A device for fixing a laboratory rodent for inhalation anesthesia, the device comprising:
a body comprising a cavity into which at least a part of a rodent including the nose of the rodent is insertable; and
a gas supply pipe inserted into the cavity for supplying an anesthetic gas to the rodent, the gas supply pipe comprising a fixing part on an end portion thereof for fixing incisors of the rodent and an opening formed at a position forward from the fixing part for discharging the anesthetic gas,
wherein the position of the opening relatively to the nose of the rodent is constantly maintained.

2. The device of claim 1, wherein a lower portion of the cavity in which the fixing part and the opening are located has a diameter greater than that of an opposite upper portion of the cavity.

3. The device of claim 2, wherein the lower portion of the cavity has a cone shape.

4. The device of claim 1, wherein a packing member is provided on a lower portion of the cavity in which the fixing part and the opening are located, so as to make tight contact with the rodent and prevent leakage of the anesthetic gas, exhaled gas of the rodent, and remaining gas.

5. The device of claim 1, wherein a flat support surface is formed on at least a portion of the body.

6. The device of claim 1, further comprising a fixing member to detachably fix the gas supply pipe to the body.

7. The device of claim 1, wherein the opening and the fixing part are provided as parts of a replaceable tip that is attachable to the gas supply pipe and detachable from the gas supply pipe.

8. The device of claim 1, further comprising a discharge tube connected to the cavity for drawing in exhaled gas of the rodent or remaining gas and discharging the exhaled gas and the remaining gas to an outside area.

9. The device of claim 8, wherein a discharge pipe of the discharge tube extends to a lower portion of the cavity.

10. The device of claim 8, wherein a connector is provided on an end portion of the discharge tube for connection with a monitoring device that monitors breathing of the rodent.
